(19) United States
(12) Patent Application Publication
(10) Pub. No.: US 2018/0142209 A1
(43) Pub. Date: May 24, 2018

(54) NATURAL KILLER CELL PRIMING COMPOSITION

(71) Applicant: UCL BUSINESS PLC, London (GB)

(72) Inventors: Mark W. LOWDELL, London (GB); May S. SABRY, London (GB)

(21) Appl. No.: 15/575,939

(22) PCT Filed: May 20, 2016
(86) PCT No.: PCT/GB2016/051459

§ 371 Date: Nov. 21, 2017

(30) Foreign Application Priority Data
May 21, 2015 (GB) 1508722.4

(51) Int. Cl. C12N 5/0783; A61K 39/00

(52) U.S. Cl. CPC C12N 5/0646; A61K 39/0011; A61K 2039/5154; C12N 2501/50; C12N 2501/53

(57) ABSTRACT

The present invention relates to a composition for priming a human Natural Killer (NK) cell. The present invention provides a natural killer (NK) cell priming composition which comprises (i) a CD2 ligation agent; (ii) an NKp46 ligation agent; and (iii) an LFA-1 ligation agent. The present invention also provides an NK-cell priming substrate which comprises (i) a CD2 ligation agent; (ii) an NKp46 ligation agent; and (iii) an LFA-1 ligation agent.

### FIELD OF THE INVENTION

The present invention relates to a composition for priming a human Natural Killer (NK) cell. Following priming, the NK cell may have the capacity to lyse an NK-resistant cancer cell.

### BACKGROUND TO THE INVENTION

A number of cancers are, at present, incurable. For others, chemotherapy is only partially effective and a significant proportion of patients relapse following treatment. Some haematological malignancies are treatable by hematopoietic stem cell transplantation (HSCT), but fewer than 30% of patients requiring HSCT have a suitable donor and are the requisite age.

Natural Killer (NK) cells are a subset of peripheral blood lymphocytes which can spontaneously lyse certain tumour cells. The use of NK cells in adoptive tumour immunotherapy has been proposed, and there has been interest in the in vitro or ex vivo stimulation of NK cells to increase their capacity to lyse tumour cells.

The discovery of interleukin-2 (IL-2) and its role in NK-cell activation in the 1980's led to considerable interest in the use of lymphokine-activated killer (LAK) cells in tumour immunotherapy. The results of these trials were, however, largely disappointing. In a study investigating the effect of administering autologous LAK cells to patients along with IL-2, fewer than 20% of patients responded (Rosenburg et al (1987) N. Engl. J. Med. 316: 889-897). In studies using daily IL-2 administrations to cancer patients along with chemotherapy and autologous HCT, it was shown that, although IL-2 significantly expanded the number of circulating NK cells in vivo, the cells are not maximally cytotoxic according to an in vitro assay (Miller et al (1997) Biol. Blood Marrow Transplant. 3: 34-44).

Resting human NK cells require at least two activating signals before commitment to cytokine secretion and/or target cell lysis. It has been shown that these two signals can be divided into discrete “priming” and “triggering” events, with the priming signal being provided either by an activating cytokine, such as IL-2, or conjugation to a tumour cell expressing an appropriate intensity and combination of signals (North et al (2007) J. Immunol. 178:85-94).

WO 2006/097743 describes a method for activating an NK-cell by contacting the NK-cell in vitro with a preparation of the tumour cell line CTV-1, which primes resting NK-cells but fails to trigger lysis.

These tumour-primed NK-cells are then capable of killing a variety of previously resistant tumours as well as secreting a milieu of activating cytokines and chemokines that aid in the mounting of an effective immune response. The priming procedure involves co-culturing of CTV-1 lysate with freshly isolated NK-cells, followed by lysate removal prior to re-infusion into the patient.

Various other leukemia cell lines have been shown which prime NK cells but are resistant to lysis, including MV-411 and SEM (Sabry et al (2011) J. Immunol. 187:6227-6234).

However, in producing tumour-primed NK-cells, the removal of the tumour cell lysate is technically demanding and leads to significant loss of the NK-cells. In addition, there are safety issues associated with the use of tumour cell preparations.

There is thus a need for an alternative ex vivo method to prime NK-cells which avoids the safety issues and technical complications of using a tumour cell preparation. Preferably, these primed NK-cells are still capable of killing a variety of previously resistant tumours.

### DESCRIPTION OF THE FIGURES

FIG. 1—Investigating bead stimulation of NK cells.

FIG. 2—Investigating the optimum ratio of NK cells to beads.

FIG. 3—Investigating the effect of bead removal on NK cells.

### SUMMARY OF ASPECTS OF THE INVENTION

It has been shown that CD2-ligation is one of the mechanisms whereby resting human NK (rNK) cells are primed to activate. CTV-1 causes CD2 ligation through expression of CD15, which can bind CD2 (Sabry et al (2011) J. Immunol. 187:6227-6234).

However, CD15 alone is insufficient to prime rNK since normal human myeloid cells which constitutively express CD15 do not prime rNK.

The present inventors have now found that it is possible to prime rNK-cells by the simultaneous ligation of (i) CD2; (ii) NKp46; and (iii) LFA-1 on rNK-cells.

Thus in a first aspect, the present invention provides a natural killer (NK) cell priming composition which comprises (i) a CD2 ligation agent; (ii) an NKp46 ligation agent; and (iii) an LFA-1 ligation agent.

The CD2 ligation agent may be an anti-CD2 antibody.

Alternatively, the CD2 ligation agent may be an oligonucleotide aptamer.

The NKp46 ligation agent may be an anti-NKp46 antibody.

Alternatively, the NKp46 ligation agent may be an oligonucleotide aptamer.

The LFA-1 ligation agent may be an anti-LFA-1 antibody.

Alternatively, the LFA-1 ligation agent may be an oligonucleotide aptamer.

In a second aspect, the present invention provides an NK-cell priming substrate which comprises a CD2 ligation agent, an NKp46 ligation agent and a LFA-1 ligation agent as defined in the first aspect of the invention.

The substrate may, for example, be a two-dimensional surface (e.g. filter, plate, well, flask, bag, or the like) or a three-dimensional surface (e.g. bead, nanoparticle or the like), coated with a CD2 ligation agent, an NKp46 ligation agent and an LFA-1 ligation agent.

The bead may be an anti-biotin bead.

In a third aspect, the present invention provides a method of priming NK-cells which comprises the step of contacting human resting NK (rNK) cells with:
an NK-cell priming composition according to the first aspect of the invention; or
an NK-cell priming substrate according to the second aspect of the invention.

The rNK-cell may be obtained from a patient.

In a fourth aspect, the present invention provides a population of primed NK-cells produced by a method according to the third aspect of the invention.

The primed NK-cells may be characterised by an increased expression of CD69.

The primed NK-cells may be characterised by an increased ability to kill NK-insensitive Raji cells compared to un-primed resting NK cells.

The primed NK-cells may maintain its primed state following removal of the NK-cell priming composition or substrate.

The primed NK-cells may maintain its primed state following cryopreservation.

In a fifth aspect, the present invention provides a pharmaceutical composition for administration to a patient comprising a population of primed NK-cells according to the fourth aspect of the invention in a pharmaceutically acceptable medium.

In a sixth aspect, the present invention provides a method of treating cancer comprising the step of administering a pharmaceutical composition according to the fifth aspect of the invention to a subject.

In a seventh aspect, the present invention provides a pharmaceutical composition according to the fifth aspect of the invention for use in the treatment of cancer.

In an eighth aspect, the present invention provides the use of a population of primed NK-cells according to the fourth aspect of the invention in the manufacture of a pharmaceutical composition for the treatment of cancer.

In a ninth aspect, the present invention provides a method of treating cancer in a subject which comprises the step of administering an NK-cell priming composition according to the first aspect of the invention, or an NK-cell priming substrate according to the second aspect of the invention to the subject.

In a tenth aspect, the present invention provides a kit for preparing a composition according to the first aspect of the invention which comprises (i) a CD2 ligation agent, (ii) an NKp46 ligation agent and (iii) an LFA-1 ligation agent.

Because the method of the present invention causes priming of an NK-cell via ligation of three cell surface receptors by ligation agents, the method is entirely “synthetic” and does not rely on the presence of an activating tumour cell (e.g. CTV-1) or cell membrane preparation thereof. The invention replaces the tumour cell with an artificial NK-cell priming reagent using a solid phase presentation system (such as a filter, plate, well, flask, bag or other two dimensional surface, beads, nanoparticles or liposomes). The method of the invention therefore overcomes the disadvantages associates with the tumour cell activation method, as outlined in the previous section.

WO 2013/175237 describes a method of priming NK-cells by contacting the NK-cell in vitro with a composition comprising a CD2 ligation agent and an NKG2D ligation agent. It has also been found that NK cells activated by the method of the present invention are more effective at priming NK-cells than the NK cells described in WO 2013/175237.

### DETAILED DESCRIPTION

### Natural Killer (NK) Cell

Human NK cells are a subset of peripheral blood lymphocytes defined by the expression of CD56 and the absence of the T cell receptor (CD3). They recognise and kill transformed cell lines without priming, in an MHC-unrestricted fashion.

NK cells represent the predominant lymphoid cell in the peripheral blood for many months after clinical allogeneic or autologous stem cell transplant and they have a primary role in immunity to pathogens during this period (Reittie et al (1989) Blood 73: 1351-1358; Lowdell et al (1998) Bone Marrow Transplant 21: 679-686). The role of NK cells in engraftment, graft-versus-host disease, anti-leukemia activity and post-transplant infection is reviewed in Lowdell (2003) Transfusion Medicine 13:399-404.

Human NK cells mediate the lysis of tumour cells and virus-infected cells via natural cytotoxicity and antibody-dependent cellular cytotoxicity (ADCC).

Human NK are controlled by positive and negative cytolytic signals. Negative (inhibitory) signals are transduced by C-lectin domain containing receptors CD94/NKG2A and by some Killer Immunoglobulin-like Receptors (KIRs). The regulation of NK lysis by inhibitory signals is known as the “missing self” hypothesis in which specific HLA-class I alleles expressed on the target cell surface ligate inhibitory receptors on NK cells. The down-regulation of HLA molecules on some tumour cells and some virally infected cells (e.g. CMV) lowers this inhibition and concomitantly reduces the level of activating signals required to initiate lysis. Artificial NK priming overcomes the inhibitory signals, allowing primed NK cells to lyse tumour cells expressing normal levels of HLA molecules.

Inhibitory receptors fall into two groups, those of the Ig-superfamily called Killer Immunoglobulin-like Receptors (KIRs) and those of the lectin family, the NKG2, which form dimers with CD94 at the cell surface. KIRs have a 2- or 3-domain extracellular structure and bind to HLA-A, -B or -C. The NKG2/CD94 complexes ligate HLA-E.

Inhibitory KIRs have up to 4 intracellular domains which contain ITIMs and the best characterized are KIR2DL1, KIR2DL2 and KIR2DL3 which are known to bind HLA-C molecules. KIR2DL2 and KIR2DL3 bind the group 1 HLA-C alleles whilst KIR2DL1 binds to group 2 alleles. Certain leukemia/lymphoma cells express both group 1 and 2 HLA-C alleles and are known to be resistant to NK-mediated cell lysis

As regards positive activating signals, ADCC is mediated via CD16 and a number triggering receptors involved in natural cytotoxicity have been identified, including CD2, CD38, CD69, NKRP-1, CD40, B7-2, NK-TR, NKp46, NKp30 and NKp44. In addition, several KIR molecules with short intracytoplasmic tails are also stimulatory. These KIRs (KIR2DS1, KIR2DS2 and KIR2DS4) are known to bind to HLA-C; their extracellular domains being identical to their related inhibitory KIRs. The activatory KIRs lack the ITIMs and instead associate with DAP12 leading to NK cell activation. The mechanism of control of expression of inhibitory versus activatory KIRs remains unknown.

Donor NK cells may be HLA-KIR matched or mismatched. The present inventors have shown that the degree of matching between the NK cells and target tumour cells is of no significance.

The NK cells activated by the method of the present invention may be autologous or allogeneic NK cells.

“Autologous” NK cells are cells derived from the patient. “Allogeneic” NK cells are derived from another individual, having non-identical gene at one or more loci. If the NK cells are derived from an identical twin, they may be termed “syngeneic”.

### Priming

Resting human NK cells require a two-stage activation process, involving a “priming” and a “triggering” step. NK-sensitive tumours provide both priming and triggering signals, leading to lysis. NK-resistant tumours evade lysis, mostly by failure to prime. However, there are some tumour cells, such as the tumour cell line CTV-1, which have the capacity to prime tumour cells but fail to trigger lysis.

It has previously been shown that such cells can be used to prime or activate NK cells, such that it can go on to lyse a target cell which is resistant to lysis by an equivalent unstimulated NK cell.

The terms “priming”, “activating” and “stimulating” NK cells are used synonymously in this document to mean rendering a resting NK into a state such that can lyse a target cell which is normally resistant to NK cell lysis. The target cell may be an NK resistant tumour cell. Raji and Daudi cell lines are useful models for NK-resistant tumours.

The composition of the present invention is capable of priming an NK cell to achieve the same activation state as treatment with an activating tumour cell preparation of, for example, CTV-1 cells.

Primed or activated NK cells have a characteristic phenotype, which distinguishes them from resting NK cells.

In preferred embodiments, incubation of NK cells with the subject ligation agents as described herein causes rapid upregulation of CD69 on the NK cells. In addition to CD69, the IL-2 receptor, CD25, is also upregulated. Accordingly, in a preferred embodiment the subject ligation agents produce an activated NK cell population that is CD69+ and/or CD25+.

### Ligation Agents

The composition of the present invention comprises at least three ligation agents: a CD2 ligation agent, an NKp46 ligation agent and an LFA-1 ligation agent.

A ligation agent is an entity which binds and activates a receptor.

A receptor is a cell-associated protein that binds to a bioactive molecule (the “ligand”) and mediates the effect of the ligand on the cell. Binding of ligand to receptor results in a change in the receptor (and, in some cases, receptor multimerization, i.e., association of identical or different receptor subunits) that causes interactions between the effector domain(s) of the receptor and other molecule(s) in the cell. These interactions in turn lead to alterations in the metabolism of the cell.

The ligation agent may be a natural or synthetic ligand for the receptor. The ligation agent may trigger the same intracellular effect as the natural ligand. The ligation agent may be a binding agent such as an antibody.

The ligation agent may be an agonist for the receptor, or an analog or derivative thereof including, e.g., a fusion of the agonist with another protein or compound.

The ligation agent may be an antibody or an antigen-binding fragment thereof, including, e.g., a monoclonal or polyclonal antibody, a tetrabody, a nanobody, a chimeric antibody, a deimmunized antibody, a humanized antibody or a human antibody. In particular embodiments of the present invention the antigen binding fragment is selected from the group consisting of F(ab)2, F(ab′)2, Fab, Fab′, Fd, Fv, single-chain Fv, and disulfide-linked Fvs (dsFv). The term “antibody” includes antibody-like molecules with alternative scaffolds such as DARPins and other repeat protein scaffolds and domain antibodies (d(Ab)s).

The ligation agent may be an aptamer. “Aptamers” as used herein may be oligonucleotide or peptide aptamers. Aptamers are molecules which bind to a specific target molecule.

Methods of generating aptamers are well known in the art. For example, aptamers can be generated through systematic evolution of ligands by exponential enrichment (SELEX) to bind to various targets of interest. Aptamers can also be selected from surface-display technologies such as phage display, mRNA display, ribosome display etc.

The ligation agents can be modified, e.g., by the covalent attachment of any type of molecule as long as such covalent attachment permits the agonist or antibody to retain its activation of the receptor. For example, but not by way of limitation, suitable derivatives and analogs of the ligation agents include those that have been further modified, e.g., by glycosylation, acetylation, pegylation, biotinylation, phosphorylation, amidation, derivatization by known protecting/blocking groups, proteolytic cleavage, linkage to a substrate or other protein, etc. Any of numerous chemical modifications can be carried out by known techniques, including, but not limited to specific chemical cleavage, acetylation, formylation, etc. Additionally, the analog or derivative can contain one or more unnatural amino acids, or have a modification (e.g., substitutions, deletions or additions) in amino acid residues that interact with Fc receptors.

### CD2 Ligation Agent

CD2 is a cell adhesion molecule found on the surface of T cells and NK cells. It acts as a costimulatory molecule.

It has been shown that, during tumour mediated NK priming, CD2 on the NK cell binds to a ligand within CD15 on the tumour cell (Sabry et al (2011) J. Immunol. 187:6227-6234). Blockade of CD15 on tumour cells has been shown to significantly inhibit priming of NK cells. Also, NK-resistant Raji cells become susceptible to NK lysis following transfection and expression of CD15. Crucially, this CD2 ligation is different from the common ligation by CD58 and uses a different ligation site within CD2 leading to the downstream signalling pathway described above. CD2 ligation with CD58 or with antibodies which mimic CD58 binding do not prime rNK cells.

The CD2 ligation agent may comprise the CD2L site of CD15. The ligand for CD2 of CD15 is a carbohydrate structure which is closely associated with, yet distinct from CD15 (Warren et al (1996) J. Immunol. 156:2866-2873). The carbohydrate structure is Gal-β1-4 GlcNAc α1-3Fuc. The ligation agent may comprise this carbohydrate structure optionally in association with all or a part of CD15.

The CD2 ligation agent may be an anti-CD2 antibody.

Various anti-human CD2 antibodies are known, such as clone RPA-2.10 (Bryceson et al (2006) Blood 107:159-166), clone LT2 (Sigma-Aldrich) and OKT11 (Sabry et al (2011 as above).

The anti-CD2 antibody may be biotinylated. Biotinylation is the process of covalently attaching biotin by either chemical or enzymatic means.

The CD2 ligation agent may be an oligonucleotide aptamer.

CD2 ligation through this binding site activates the CD3ζ-LAT-Stat5 pathway in NK cells. The cytoplasmic CD16/CD3ζ complex interacts with the intracellular domain of CD2, leading to the phosphorylation of linker for activation of T cells (LAT). Ligation of CD2 leads to phosphorylation of Stat5 and upregulation of CD25 and CD69.

The CD2 ligation agent may cause activation of the CD3ζ-LAT-Stat5 pathway. This may be detected by monitoring phosphorylation of LAT and/or Stat5; by examining the levels of CD25 and/or CD69; or by investigating the production of IFNγ.

### NKp46 Ligation Agent

NKp46 is a known lysis receptor on NK-cells. It acts as a co-stimulatory molecule for NK-cell activation.

Lysis receptors such as NKp46 are known to be able to trigger NK-mediated lysis of various tumour cell types, through direct engagement of the membrane ligands expressed by said tumour cells.

NKp46 is a transmembrane type I glycoprotein. It contains 2 immunoglobulin domains and transmembrane domain. The transmembrane domain contains a positively charged arginine residue and is known to associate with the TCR signalling molecule.

The NKp46 ligation agent may be an anti-NKp46 antibody.

Various anti-human NKp46 antibodies are known, such as clone 9E2 (Fisher Scientific).

The anti-NKp46 antibody may be biotinylated. Biotinylation is the process of covalently attaching biotin by either chemical or enzymatic means.

The NKp46 ligation agent may be an oligonucleotide aptamer.

### LFA-1 Ligation Agent

LFA-1, also known as lymphocyte function-associated antigen 1 is an integrin adhesion molecule found on T-cells, B-cells, macrophages and neutrophils. Integrins are transmembrane receptors which are obligate heterodimers of two different chains, known as the α and β subunits.

LFA-1 comprises the α-chain CD11a, and the β-chain CD18. It is known to bind to ICAM-1 which is found on antigen-presenting cells.

The LFA-1 ligation agent may be an anti-LFA-1 antibody.

Various anti-human LFA-1 antibodies are known, such as clone REA378 (Miltenyi Biotec).

The anti-LFA-1 antibody may be biotinylated. Biotinylation is the process of covalently attaching biotin by either chemical or enzymatic means.

The LFA-1 ligation agent may be an oligonucleotide aptamer.

### Substrate

The second aspect of the present invention provides a substrate which comprises a CD2 ligation agent, and NKp46 ligation agent and an LFA-1 ligation agent. The ligation agents may be attached to the surface of the substrate.

The substrate may be used to activate rNK cells, by bringing the rNK cells into contact with the substrate such that ligation of CD2, NKp46 and LFA-1 on the NK cells occurs.

One of the advantages of using a solid substrate is it facilitates removal of the activating agents (namely the CD2, NKp46 and LFA-1 ligation agents) following NK cell activation. Once the substrate is removed, the resulting preparation should comprise activated NK cells in a relatively pure form (i.e. without the CD2, NKp46 and LFA-1 ligation agents).

Where the ligation agents are antibodies, it is possible to use substrates coated with a “second-layer” antibody (i.e. an antibody reactive with the ligation agent or a covalent moiety on the ligation agent) in order to attach the ligation agents.

The substrate may present a two dimensional surface, such as a filter, plate, well, flask, roller bottle, capillary, bag and the like; or a three-dimensional surface, such as a bead, liposome or microparticle. Magnetic/paramagnetic beads or particles may be used to aid separation. The substrate material employed in the present invention may be of any suitable material and may be a porous or a non-porous support. Preferably, the substrate is a solid support, particle or bead.

Beads, or magnetic beads, are spherical polymer particles which can be coated with additives including antibodies or other proteins such as streptavidin. These antibodies may bind further secondary antibodies to increase sensitivity. These beads can attach to receptors or other proteins of interest through the binding of the coated antibody, linked antibody, or protein. An advantage of using magnetic beads is the ease of separation. The use of a magnetic field will preferentially capture the magnetic beads and remove them and any coated additives from a mixture.

In one embodiment, the substrate is comprised of a cross-linked carbohydrate material, such as agarose, agar, cellulose, dextran, chitosan, konjac, carrageenan, gellan, alginate etc. The substrate may easily be prepared according to standard methods, or is a commercially available product, such as DYNABEADS™ (Life Technologies, California, USA), Anti-Biotin MACSIBead Particles (Miltenyl Biotec, Cologne, Germany) or SEPHADEX™ or SEPHAROSE™ FF (Amersham Biosciences AB, Uppsala, Sweden). In an alternative embodiment, the substrate is comprised of cross-linked synthetic polymers, such as styrene or styrene derivatives, divinylbenzene, acrylamides, acrylate esters, methacrylate esters, vinyl esters, vinyl amides, or may be an inorganic material such as glass or silica. Preferably, the polymer is selected from the group consisting of polystyrene, polypropylene, polyvinyltoluene, polyacrylamide, polyacrylonitrile and polycarbonate. Solid supports of such polymers are easily produced according to standard methods, see e.g. “Styrene based polymer supports developed by suspension polymerization” (Arshady, R., Chimica e L′Industria, (1988), 70(9), 70-75).

The invention also provides a method for making a substrate according to the second aspect of the invention which comprises the step of attaching a CD2 ligation agent, an NKp46 ligation agent and an LFA-1 ligation agent to a substrate. The agents may be directly attached, or attached via another entity, such as an antibody or linker.

### Vesicle

Described herein are vesicles which comprise a CD2 ligation agent, an NKp46 ligation agent and an LFA-1 ligation agent. The ligation agents are displayed on the surface of the vesicle such that they can interact with CD2, NKp46, and LFA-1 on the surface of rNK cells.

The vesicle may be a membranous vesicle. A liposome is an artificial vesicle composed of a lipid bilayer.

It is also possible to express proteins within liposomes by providing the liposomes with the necessary machinery to transcribe and translate the relevant gene.

### Activation Method

The present invention also provides a method for priming NK-cells which comprises the step of contacting human resting NK (rNK) cells with:
an NK-cell priming composition according to the present invention; or
an NK-cell priming substrate according to the present invention.

The step of contacting rNK cells with an NK-cell priming composition or substrate according to the present invention may occur in vitro.

In accordance with the present invention, NK cells may be activated by the one of the above methods alone, or in combination with another NK cell activation technique.

For example, U.S. Pat. No. 7,435,596 describes the expression of chimeric receptors on NK cells to enhance their capacity to kill target cells. The NK cells of the present invention may express a chimeric receptor comprising an anti-CD19 receptor and a signalling domain. The signalling domain may, for example be CD3 ζ or DAP10.

The chimeric receptor may also comprise the costimulatory molecule 4-1BB.

The resting NK cell may be autologous or allogeneic.

Allogenic NK cells may be obtained from peripheral blood from a donor individual. Allogeneic peripheral blood mononuclear cells may be collected by standard techniques (e.g. conventional apheresis). To minimize the possibility of graft versus host disease and immune mediated aplasia, allogeneic cells may be depleted of T cells. For example, the cell preparation may be depleted of CD3+ T-cells using microbeads conjugated with monoclonal mouse anti-human CD3 antibody and a cell selection device (such as the Miltenyi Biotec CliniMACS® cell selection device).

However, NK cells produced by such “negative selection” procedures alone do not have a high degree of purity and may be contaminated with T and B cells.

In order to reduce contamination, it is possible to obtain an NK cell preparation by direct immunomagnetic separation, for example on the basis of CD56 expression. To further reduce T cell contamination, the product may be depleted for CD3+ cells (for example using CD3 FITC and anti-FITC beads).

Prior to activation by the method of the invention, the NK cell preparation may comprise at least 80%, at least 90%, at least 95% or at least 98% CD56+ cells.

Prior to activation by the method of the invention, the NK cell preparation may comprise less than 15%, less than 10%, less than 5% or less than 3% CD3+ cells.

### Primed NK-Cells

The present invention also provides a population of primed NK-cells produced by a method according to the present invention.

The population of primed NK-cells may be further characterised by an increased expression of CD69.

The population of primed NK-cells may be further characterised by an increased ability to kill NK-insensitive Raji cells compared to un-primed resting NK cells. The Raji cell line is a well known human cell line. Ragi cells are lymphoblastoid cells originally derived from a Burkitt lymphoma.

The population of primed NK-cells may maintain its primed state following removal of the NK-cell priming composition or substrate.

The population of primed NK-cells may maintain its primed state following cryopreservation.

Being in a primed state may be characterised by expression of the CD69 activation marker.

Being in a primed state may be characterised by a capacity to kill NK-insensitive Raji cells in a 4 hour cytotoxicity assay.

### Pharmaceutical Composition

The present invention also provides a pharmaceutical composition comprising a population of primed NK-cells activated by a method of the invention.

The composition may comprise or consist essentially of autologous and/or allogeneic NK-cells.

Allogeneic NK cells may be HLA mismatched.

The composition may also comprise the CD2 ligation agent, NKp46 ligation agent and LFA-1 ligation agent; or a substrate or vesicle comprising the CD2 ligation agent, NKp46 ligation agent and LFA-1 ligation agent.

The CD2 ligation agent, NKp46 ligation agent and LFA-1 ligation agent may be the only activation the NK cells receive, or there may be further activation steps. The NK cells may or may not also be non-specifically activated by IL-2 (for example by incubation of the cells in medium supplemented with IL-2). Alternatively, the cells may be activated in the absence of IL-2, but IL-2 may be used for the ex vivo expansion of stimulated cells.

### Method of Treatment

The composition of the present invention may be used in medicine. For example, the composition may be used to treat or prevent cancer or infection in a subject.

The composition comprising a population of primed NK-cells may be manufacture of a medicament for the treatment of cancer or infection.

The composition may be administered to the subject by any suitable method known in the art, for example, intravenous infusion.

The present invention also provides a method for treating a subject in need of same, which comprises the following steps:
(i) priming a resting NK-cell in vitro by a method according to the present invention; and
(ii) administering the activated NK cell to the subject.

The method may be for treating a disease in the subject. The disease may be cancer or an infection.

The composition may be used to treat a subject in need of same. The procedure is low-risk and particularly suitable for cancer patients for whom intensive cancer treatments are precluded (for example, elderly patients). It also provides an alternative for patients (with, for example, lymphoma, myeloma or AML) who lack a suitable donor for allogeneic stem cell transplantation.

Prior to treatment with the composition, the patient may receive some pre-treatment, for example, to de-bulk the tumour and/or immunosuppress the patient. This may be achieved, for example, by chemotherapy, radiotherapy or a combination thereof.

It is possible to obtain primary tumour cells from patients at time of diagnosis and to cryopreserve these as viable single cell suspensions. It is thus possible for a composition according to the invention to be tested in vitro against patient blasts. This could be done before embarking on a treatment regime, to gauge the suitability of the approach. The correlation of the results of the in vitro study and the corresponding clinical response to treatment may also be investigated.

### Disease

The activated NK cell composition may be used to treat or prevent a disease or medical condition.

The disease may be a cancer. There are about 200 different types of cancer. Lists of types of cancer are available (for example, see http://www.acor.org/types.html or http://www.cancerresearchuk.org).

Some more common cancers include leukaemia (acute and chronic), bladder cancer, bone cancer (osteosarcoma), Bowel (colorectal cancer), brain cancer, breast cancer, cervical cancer, oesophageal cancer, Hodgkin's lymphoma, kidney cancer, liver cancer, lung cancer, mesothelioma, multiple myeloma, non-Hodgkin's lymphoma, ovarian cancer, pancreatic cancer, penile cancer, prostate cancer, skin cancer (melanoma and non-melanoma) soft tissue carcinoma, gastric cancer, testicular cancer, thyroid cancer and endometrial cancer.

The primed NK-cell composition produced by the method of the present invention may be useful to treat any cancer which is accessible to NK-cells.

In particular the cancer may be a haematological malignancy, such as leukaemia (AML); myeloma; Lymphoma.

Myeloma is an incurable and fatal malignancy. NK activity against myeloma plasma cells is documented in vitro and enhanced NK activity against autologous myeloma cells has been shown to correlate with response to treatment with Thalidomide derivatives. Myeloma patients are generally young and fit enough to undergo autologous haematopoietic stem cell transplantation and could readily undergo a less invasive procedure such as the one provided by the present invention.

Post-transplant lymphoproliferative disease (PTLD) is a serious and relatively common complication after solid organ transplantation and T cell immunotherapy is currently under trial but with little success. Therapy using NK cells activated according to the present invention therapy would be easy and safe in this group of patients.

In addition the composition may be used to treat solid tumours such as breast cancer.

The procedure is particularly suitable to treat “NK-resistant” tumours. Normal NK cells can spontaneously lyse some human tumours, but many other tumours are NK-resistant. “NK-resistant” as used herein, therefore, indicates tumour cells resistant to lysis by normal NK cells which have not been stimulated with a ATOP or by the method according to the present invention.

As explained above, inhibition of NK-mediated lysis is controlled by expression of specific MHC class I molecules on the target cell surface, particularly HLA-C. There are two distinct groups of HLA-C alleles with regard to NK cell recognition. Some tumours express both types of HLA-C allele, which is thought to make them resistant to NK-mediated lysis. “NK resistant” cells may, therefore express both groups of class I allele. Some leukemia/lymphoma-derived cell lines, such as Raji and Daudi express both types of HLA-C allele, making them useful models for NK-resistant tumour cells in vivo.

Common infections that may be treated with a cellular composition comprising the activated NK cells as described herein include viral infections such as, e.g., hepatitis type A virus, hepatitis type B virus, hepatitis type C virus, etc.; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Barr virus; poxviruses, such as variola (smallpox) and vaccinia virus; RNA viruses, including but not limited to human immunodeficiency virus type I (HIV-1), human immunodeficiency virus type II (HIV-11), human T-cell lymphotropic virus type I (HTLV-I), and human T-cell lymphotropic virus type II (HTLV-II); influenza virus; measles virus; rabies virus; Sendai virus; picornaviruses such as poliomyelitis virus; coxsackieviruses; rhinoviruses; reoviruses; togaviruses such as rubella virus (German measles) and Semliki forest virus; arboviruses; rinderpest; echovirus; rotavirus; respiratory syncytial virus; echinovirus; huntavirus; mumps virus; measles virus; rubella virus; polio virus; coronavirus; and combinations thereof.

### Kit

The present invention also provides a kit for preparing a NK-cell activating composition according to the present invention.

The present invention also provides a kit for use in a method for activating an NK cell according to the method of present invention.

The kit may comprise (i) a CD2 ligation agent; (ii) an NKp46 ligation agent; and (iii) an LFA-1 ligation agent or a precursor thereof. For example, where the CD2 ligation agent and/or NKp46 ligation agent and/or LFA-1 ligation agent is/are a protein, the kit may comprise a protein-encoding gene.

The present invention also provides a method for preparing a composition according to the present invention, which comprises the step of combining (i) a CD2 ligation agent; (ii) an NKp46 ligation agent; and (iii) an LFA-1 ligation agent.

The present invention also provides the use of the kit in a method for preparing a substrate according to the present invention, which method comprises the step of attaching or immobilising (i) a CD2 ligation agent; (ii) an NKp46 ligation agent; and (iii) an LFA-1 ligation agent on a substrate.

The present invention also provides the use of a kit in a method for preparing a vesicle according to the present invention, which method comprises the step of causing (i) a CD2 ligation agent; (ii) an NKp46 ligation agent; and (iii) an LFA-1 ligation agent to be expressed or present on the surface of a vesicle.

The kit may also comprise instructions for use.

The invention will now be further described by way of Examples, which are meant to serve to assist one of ordinary skill in the art in carrying out the invention and are not intended in any way to limit the scope of the invention.

### EXAMPLES

### Example 1—Bead Stimulation of NK-Cells

Different ligand combinations (human anti-CD2/NKp46/NKG2D/or LFA-1) were used to coat different types of beads (Dynabeads sheep anti-mouse IgG, Dynabeads Protein A/G or MACSibead particles) with varying stimulator-responder ratios in order to design a bead stimulation system for optimised NK cell anti-tumour activity.

Anti-Biotin beads of 3.5 μm diameter (MACSibead Particles, Miltenyi Biotec) were coated with different combinations of biotinylated antibodies against human CD2, NKG2D, NKp46 and LFA-1. NK cells were co-incubated with the loaded beads (bead:cell ratio of 1:2) and analysed for primed activity. Freshly isolated NK cells from normal healthy volunteers were incubated alone, with CTV-1 lysate, uncoated beads, or beads coated with different combinations of anti-CD2, -NKG2D, -NKp46, and -LFA-1. Thereafter, NK cells were washed and analysed for (A) CD69 expression and (B) the ability to kill NK-insensitive Raji in a 4 hour cytotoxicity assay.

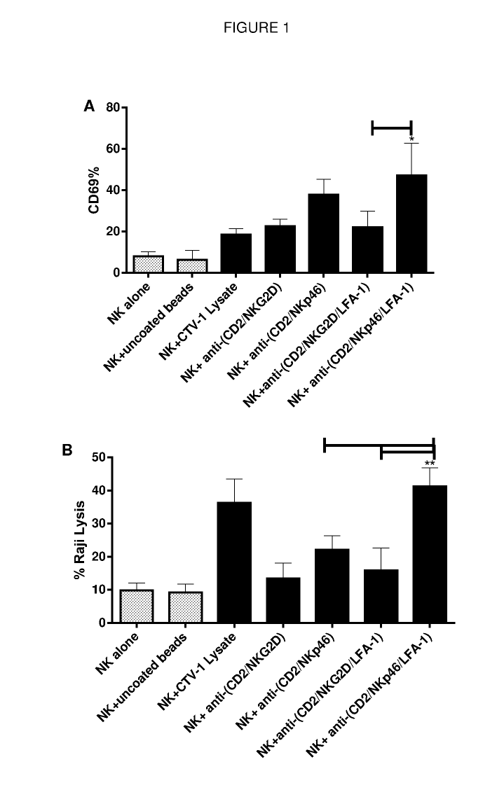

The results are shown in FIG. 1. Bars represent the mean±SD from 4 donors. Statistical significance is indicated as: *P≤1.05; **P<0.01.

Beads coated with equal amounts of anti-CD2/NKp46/LFA-1 consistently resulted in the highest percentage of CD69 expression by NK cells (mean 47.17±7.7%) and killing against NK-insensitive Raji cells (mean 41.30±5.55%). Expression of the activation marker CD69 was higher in NK cells primed with anti-(CD2/NKp46/LFA-1) beads compared to beads coated with anti-(CD2/NKp46) [mean increase 10.02±7.7%, n=4; P=NS] or anti-(CD2/NKG2D/LFA-1) [mean increase 25.1±7.8%, n=4: P=0.027]. Primed NK cell activity against Raji cells was also higher when beads coated with anti-(CD2/NKp46/LFA-1) were used compared to anti-(CD2/NKp46) [mean increase 19.22±3.5%, n=4; P=0.001] or anti-(CD2/NKG2D/LFA-1) [mean increase 25.5±4.39%, n=4: P=0.001].

The data shows that the replacement of NKG2D ligation with NKp46 together with the addition of LFA-1 does provide all of the signals required to achieve full priming of resting NK cells to express CD69 and to lyse RAJI cells to an equivalent degree to CTV-1 lysate. However, CD2/NKG2D co-ligation on its own was not, and the addition of the third ligand of LFA-1 also failed to prime the resting NK-cell to lyse the NK-insensitive Raji cell.

### Example 2—Optimising Bead to Cell Ratio

Purified NK-cells were incubated alone, with CTV-1 cells, beads coated with anti-CD2+LFA-1+NKp46 at different bead to cell ratios, or uncoated beads. NK cells were then tested for: (A) CD69 expression (B) killing activity against NK-insensitive Raji cells in a 4 h cytotoxicity assay using flow cytometry.

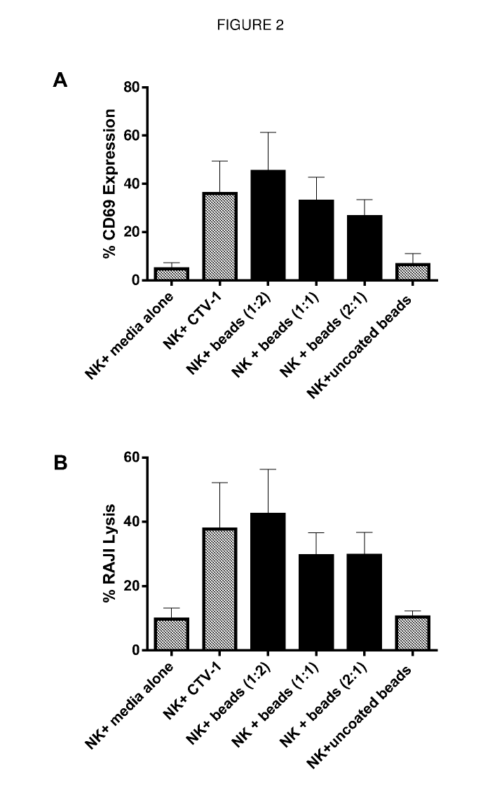

The results are shown in FIG. 2. Values represent mean±SD of 4 different donors.

These data demonstrate the optimal ratio of ligand-coated beads (CD2-NKp46-LFA1) to resting NK cells for priming as determined by CD69 expression and Raji cell killing is 1:2 (beads:cell).

### Example 3—Bead Removal Following Stimulation of NK-Cells

Different conditions were tested for optimal bead removal following NK-cell priming.

Following bead stimulation of NK-cells, the cells were transferred to a 5 ml tube and washed once with HBSS. Thereafter, cells were resuspended at a variety of densities to optimise the bead detachment and cell survival. These densities ranged from 5×10⁵ to 2×10⁶ cells per ml. The tubes were placed in the magnetic field of the MACSiMAG separator for 1, 3 and 5 minutes to determine the best time for maximal adherence to the wall of the tube with minimum cell death. The supernatant containing the MACSibead-depleted cells was carefully removed using a pipette, while retaining the tube in the magnet. The process was repeated once, twice or three times, before the cells were collected. Flow cytometric analysis was used to determine the relative loss of NK cells post bead removal.

NK cells resuspended at 1×10⁶ cells per ml following bead stimulation, placed in MACSiMAG separator for 3 minutes, with the magnetic separation process repeated twice, resulted in the optimal number of MACSibead-depleted NK cells, which was confirmed with data from 7 experiments using 4 different donors.

Numbers of beads were markedly reduced following the separation process [mean total count before wash 26667 and after three washes 452].

### Example 4—Retention of Primed Status of NK-Cells Following Depletion

The activities of primed NK-cells were investigated 6 hours post depletion to confirm that the absence of the stimulating bead did not lead to loss of primed status.

NK-cells were analysed for their expression of the NK-cell activation marker CD69 and their capacity to kill NK-resistant Raji cells in a 4 hour cytotoxicity assay.

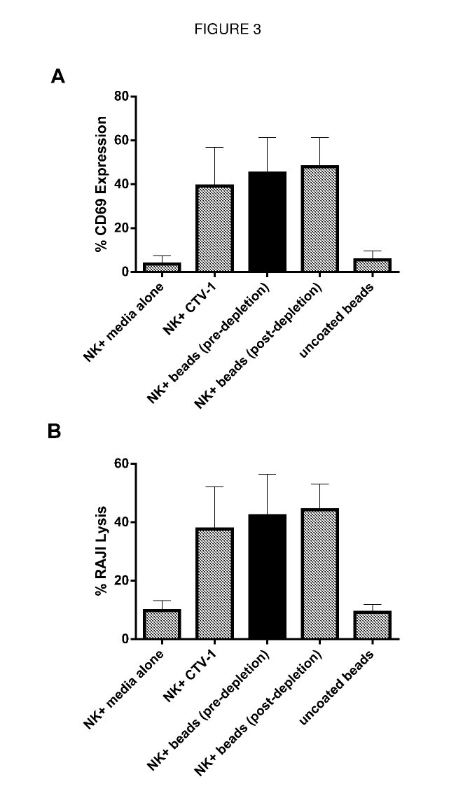

The results are shown in FIG. 3.

Results from 6 experiments using 3 different donors showed that NK cells retained their primed status for at least 6 hours after bead depletion as evidenced by their expression of the NK cell activation marker CD69 (mean 45.10±16.21%), as well as their capacity to kill NK-resistant RAJI cells in a 4 hour cytotoxicity assay (mean 45.10±16.21%).

These data support the clinical manufacture of bead primed NK since the primed state was retained for a period long enough to complete the dosing and cryopreservation steps required for commercial manufacture.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cellular immunology or related fields are intended to be within the scope of the following claims.

1. A natural killer (NK) cell priming composition which comprises (i) a CD2 ligation agent; (ii) an NKp46 ligation agent; and (iii) an LFA-1 ligation agent.

2. The NK-cell priming composition according to claim 1, wherein the CD2 ligation agent is an anti-CD2 antibody or an oligonucleotide aptamer.

3. (canceled)

4. The NK-cell priming composition according to claim 1, wherein the NKp46 ligation agent is an anti-NKp46 antibody or an oligonucleotide aptamer.

5. (canceled)

6. The NK-cell priming composition according to claim 1, wherein the LFA-1 ligation agent is an anti-LFA-1 antibody or an oligonucleotide aptamer.

7. (canceled)

8. The NK-cell priming substrate which comprises a CD2 ligation agent, an NKp46 ligation agent and a LFA-1 ligation agent according to claim 1.

9. The NK-cell priming substrate according to claim 8 which comprises a three-dimensional surface coated with the CD2 ligation agent, the NKp46 ligation agent and the LFA-1 ligation agent.

10. The NK-cell priming substrate according to claim 9, wherein the three-dimensional surface is a bead.

11. The NK-cell priming substrate according to claim 10, wherein the bead is an anti-biotin bead.

12. A method of priming NK-cells which comprises the step of contacting human resting NK (rNK) cells with:
An NK-cell priming composition according to claim 1.

13. The method according to claim 12, in which the rNK-cell is obtained from a patient.

14. A population of primed NK-cells produced by the method of claim 12.

15. The population of primed NK-cells according to claim 14, further characterised by an increased expression of CD69.

16. The population of primed NK-cells according to claim 14, further characterised by an increased ability to kill NK-insensitive Raji cells compared to un-primed resting NK cells.

17. The population of primed NK-cells according to claim 14, which maintains its primed state following removal of the NK-cell priming composition or substrate.

18. The population of primed NK-cells according to claim 14, which maintains its primed state following cryopreservation.

19. A pharmaceutical composition for administration to a patient comprising a population of primed NK-cells according to claim 14 in a pharmaceutically acceptable medium.

20. A method of treating cancer comprising the step of administering a pharmaceutical composition according to claim 19 to a subject.

21-22. (canceled)

23. A method of treating cancer in a subject which comprises the step of administering the NK-cell priming composition according to claim 1 to the subject.

24. (canceled)

25. A kit for preparing a composition according to claim 1 which comprises (i) a CD2 ligation agent, (ii) an NKp46 ligation agent and (iii) an LFA-1 ligation agent.

26. A method of treating cancer in a subject which comprises the step of administering the NK-cell priming substrate according to claim 8 to the subject.

27. A method of priming NK-cells which comprises the step of contacting human resting NK (rNK) cells with:
An NK-cell priming substrate according to claim 8.

28. The method according to claim 27, in which the rNK-cell is obtained from a patient.
